(19) United States
(12) Patent Application Publication
(10) Pub. No.: US 2005/0032135 A1
(43) Pub. Date: Feb. 10, 2005

(54) Monocarboxylate transporters expressed in cancer cells

(75) Inventors: Noa Zerangue, Sunnyvale, CA (US); Laxminarayan Bhat, Santa Clara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Xenoport, Inc., Santa Clara, CA (US)

(21) Appl. No.: 10/885,895

(22) Filed: Jul. 6, 2004

Related U.S. Application Data
(60) Provisional application No. 60/484,630, filed on Jul. 3, 2003.
(60) Provisional application No. 60/531,089, filed on Dec. 19, 2003.

(51) Int. Cl. G01N 33/574; A61K 31/195

(52) U.S. Cl. 435/7.23; 514/567

(57) ABSTRACT

MCT1 and MCT4 are expressed at higher levels in many cancer cells than comparable normal cells of the same tissue. Disclosed herein are assays for determining whether a test material/molecule is a substrate for, and/or is actively transported by, the MCT1 and/or MCT4, as well as inhibitors and substrates of MCT1 and/or MCT4, methods of isolating the same, and methods of using the same for treatment or diagnosis of cancer.

### BACKGROUND

Cancer remains the second leading cause of death in the developed world, with solid tumors of the lung, colon, breast, prostate, pancreas, ovary and testis accounting for the majority of cancer deaths. Cancer mortality rates for solid tumors have remained largely unchanged despite the many advances in understanding how solid tumors arise, diagnostic screening, and new cancer drugs.

Small molecule chemotherapeutics typically do not result in a cure for solid tumor cancer, but have clinical value in slowing disease progression, and are an important component of cancer therapy due to their efficacy against a broad range of tumor types and their ability to penetrate solid tumors. These drugs target rapidly dividing malignant cells, halting cell proliferation by interfering with DNA replication, cytoskeletal rearrangements, or signaling pathways that promote cell growth. Disruption of cell division not only slows growth but can also kill tumor cells by triggering cell death. Unfortunately, these drugs also kill normal populations of proliferating cells such as those in the immune system and gastrointestinal tract, causing strong deleterious side effects—including organ failure—that can severely limit tolerated doses and compromise effectiveness.

### SUMMARY

Disclosed herein are methods of screening agents, conjugates or conjugate moieties for activity useful for treating or diagnosing cancer. These methods entail providing a cell expressing an MCT1 or MCT4 transporter, the transporter being situated in the plasma membrane of the cell. The cell is contacted with an agent, conjugate or conjugate moiety. Whether the agent, conjugate or conjugate moiety passes through the plasma membrane via the transporter is determined. In such methods, the agent, conjugate or conjugate moiety comprises a cytotoxic or imaging component or the method also includes a step of linking the agent, conjugate or conjugate moiety to a cytotoxic or imaging component.

Disclosed herein are methods of screening an agent for pharmacological activity useful for treating cancer. These methods entail determining whether the agent binds to an MCT1 or MCT4 transporter, and contacting the agent with a cancerous cell and determining whether the agent kills or inhibits growth of the cell, killing or inhibition of growth indicating the agent has the pharmacological activity.

Disclosed herein are pharmaceutical compositions comprising a cytotoxic or imaging agent linked to a conjugate moiety to form a conjugate in which the conjugate moiety has a higher Vmax for MCT1 or MCT4 than the cytotoxic agent alone. Preferably, the agent, conjugate moiety or conjugate has a Vmax of at least about 1%, more preferably at least about 5%, more preferably at least about 10%, more preferably at least about 20%, and most preferably at least 50% of the reference substrate lactate for the MCT1 or MCT4 transporter. Therefore, agents, conjugate moieties or conjugates having Vmax's of at least 1%, 10%, 20% or 50% of the Vmax of the lactate reference substrate are disclosed herein.

Disclosed herein are methods of formulating a cytotoxic or imaging agent. These methods entail linking the cytotoxic or imaging agent to a conjugate moiety to form a conjugate, wherein the conjugate moiety has a greater Vmax for an MCT1 and/or MCT4 transporter than the agent alone. The conjugate is formulated with a pharmaceutical carrier as a pharmaceutical composition.

Disclosed herein are methods of delivering a cytotoxic or imaging agent. The methods involve administering to a patient a pharmaceutical composition comprising a cytotoxic or imaging agent linked to a conjugate moiety to form a conjugate, wherein the conjugate has a higher Vmax for an MCT1 and/or MCT4 transporter, whereby the conjugate or agent, after cleavage of the conjugate moiety, passes through the transporter into the cancerous cells in the patient.

Disclosed herein are methods of formulating a cytotoxic or imaging agent. The methods entail linking the cytotoxic or imaging agent to a conjugate moiety to form a conjugate, wherein the conjugate moiety has a greater Vmax for an MCT1 and/or MCT4 transporter than the agent alone. The conjugate is formulated with a pharmaceutical carrier as a pharmaceutical composition.

Disclosed herein are methods of delivering a cytotoxic or imaging agent. These methods entail administering to a patient a pharmaceutical composition comprising a cytotoxic or imaging agent linked to a conjugate moiety to form a conjugate, wherein the conjugate has a higher Vmax for an MCT1 and/or MCT4 transporter in which the conjugate or agent, after cleavage of the conjugate moiety, passes through the transporter into the cancerous cells in the patient.

Disclosed herein are methods of treating cancer. These methods comprise administering to a patient suffering from cancer an effective amount of an inhibitor of an MCT1 and/or MCT4 transporter, whereby the inhibitor inhibits transport through the MCT1 and/or MCT4 transporters thereby killing or inhibiting the growth of cancer cells expressing the MCT1 and/or MCT4 transporters.

Disclosed herein are methods of treating cancer. In these methods, an effective amount of an inhibitor of an MCT1 and/or MCT4 transporter is administered to a patient, whereby the inhibitor inhibits transport through MCT1 and/or MCT4 transporters thereby lowering the intracellular pH within cancer cells. Before, after or concurrently with administration of the inhibitor, one administers a chemotherapeutic agent that exhibits greater toxicity at lower intracellular pH. In combination, the inhibitor and chemotherapeutic agent kill or inhibit the growth of the cancer cells with lower intracellular pH.

Disclosed herein are methods of grading a tumor. These method entail obtaining a tumor sample from a patient. One then determines expression levels of MCT1 and or MCT4 transporters, higher expression level(s) indicating a higher grade tumor.

Disclosed herein are methods for screening an agent for capacity to inhibit a cotransporter of a proton and a substrate. These methods entail providing a cell expressing an ion channel that is inhibited by reduction in intracellular pH and the co-transporter. The cell is contacted with a known substrate of the transporter and an agent. Current is measured across the cell membrane relative to the current when the cell is contacted with the known substrate in the absence of the agent, wherein an increase in current indicates the agent inhibits transport of the substrate thereby inhibiting intracellular acidification of the cell and reducing inhibition of the ion channel.

Disclosed herein are methods for screening an agent, conjugate or conjugate moiety for capacity to be a substrate for a cotransporter of a proton and a substrate, comprising a cell expressing an ion channel that is inhibited by reduction in intracellular pH and the proton-monocarboxylate co-transporter. These methods entail contacting the cell with the agent, conjugate or conjugate moiety. Under voltage-clamp conditions, a current is measured across the cell membrane relative to the current in the absence of an agent, wherein a decrease in current indicates the agent is a substrate of the co-transporter, whereby uptake of the agent, conjugate or conjugate moiety increases intracellular acidification of the cell and inhibits of the ion channel.

Disclosed herein are cells expressing a co-transporter of protons and a substrate and a pH-sensitive ion channel, the co-transporter and ion channel being situated in the plasma membrane of the cell, wherein the co-transporter and/or ion channel is encoded by a nucleic acid transformed into the cell.

Disclosed herein are methods for measuring intracellular pH. The methods comprise providing a cell expressing an ion channel that is inhibited by reduction in intracellular pH, the ion channel being situated in the plasma membrane of the cell, monitoring current across the membrane, and determining a measure of intracellular pH from the current, wherein intracellular pH increases with increasing current.

Disclosed herein are methods for measuring flux of an organic ion co-transported with a proton by a co-transporter. These methods entail providing a cell expressing an ion channel that is inhibited by reduction in intracellular pH and the co-transporter, the ion channel and the co-transporter being situated in the plasma membrane of the cell. The cell is contacted with an organic ion. Current is monitored across the membrane. The flux of the organic ion across the cell membrane can be indirectly determined since the organic ion flux is inversely related to the measured current across the membrane.

### BRIEF DESCRIPTION OF THE FIGURES

FIG. 1 shows an LC/MS/MS (liquid chromatography tandem mass spectrometry) uptake assay for recombinant MCT1 and MCT4 transporters.

FIG. 2 shows a competition assay in which an agent competes with lactate for uptake by HEK cells.

FIG. 3 shows an assay to measure transport by MCT1 into HEK cells by measurement of intracellular pH.

FIG. 4 shows the effect of the substrate 2-thiophene glyoxylic acid on intracellular pH.

FIG. 5 shows the effect of the inhibitor sulfasalazine on intracellular pH.

FIG. 6 shows the measurement of ion current across cells expressing ROMK receptor.

FIG. 7 shows that coexpression of MCT1 and ROMK in the presence of lactate reduces ion currents.

FIG. 8 shows the transport of two nitrogen mustard compounds into Xenopus oocytes expressing human MCT1.

### DEFINITIONS

Absorption by passive diffusion refers to uptake of an agent that is not mediated by a specific transporter protein. An agent that is substantially incapable of passive diffusion has a permeability across a standard cell monolayer (e.g., Caco-2 or MDCK (Madin Darby canine kidney) cells) in vitro of less than 5×10⁻⁶ cm/sec, and usually less than 1×10⁻⁶ cm/sec in the absence of an efflux mechanism.

A “substrate” of a transport protein is a compound whose uptake into or passage through a cell is facilitated at least in part by a transporter protein.

The term “ligand” of a transport protein includes substrates or other compounds that bind to the transport protein without being taken up or transported through a cell. Some ligands by binding to the transport protein inhibit or antagonize uptake of the substrate or passage of substrate through a cell by the transport protein. Some ligands by binding to the transport protein promote or agonize uptake or passage of the compound by the transport protein or another transport protein. For example, binding of a ligand to one transport protein can promote uptake of a substrate by a second transport protein in proximity with the first transport protein.

The term “agent” is used to describe a compound that has or may have a pharmacological activity. Agents include compounds that are known drugs, compounds for which pharmacological activity has been identified but which are undergoing further therapeutic evaluation, and compounds that are members of collections and libraries that are to be screened for a pharmacological activity.

An agent is “orally active” if it can exert a pharmaceutical activity when administered via an oral route.

A “conjugate” refers to a compound comprising an agent and a chemical moiety (i.e., conjugate moiety) bound thereto, which moiety by itself or in combination with the agent renders the conjugate a substrate for active transport, for example rendering the conjugate to be a substrate for a transport protein. The chemical moiety may or may not be subject to cleavage from the agent upon uptake and metabolism of the conjugate in the patient's body. In other words, the moiety may be cleavably bound to the agent or non-cleavably bound to the agent. The bond can be a direct (i.e., covalent) bond or the bond can be through a linker. In cases where the bond/linker is cleavable by metabolic processes, the agent, or a further metabolite of the agent, is the therapeutic entity. In cases where the bond/linker is not cleavable by metabolic processes, the conjugate is the therapeutic entity. Most typically, the conjugate comprises a prodrug having a metabolically cleavable moiety, where the conjugate itself does not have pharmacological activity but the agent to which the moiety is cleavably bound does have pharmacological activity. Typically, the moiety facilitates therapeutic use of the agent by promoting uptake of the conjugate via a transporter. Thus, for example, a conjugate comprising an agent and a conjugate moiety may have a Vmax for a transporter that is at least 2, 5, 10, 20, 50 or 100-fold higher than that of the agent alone. A conjugate moiety can itself be a substrate for a transporter or can become a substrate when linked to the agent (e.g., valacyclovir, an L-valine ester prodrug of the antiviral drug acyclovir). Thus, a conjugate formed from an agent and a moiety can have higher uptake activity than either the agent or the moiety alone.

A “pharmacological” activity means that an agent exhibits an activity in a screening system that indicates that the agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

Vmax and Km of a compound for a transporter are defined in accordance with convention. Vmax is the number of molecules of compound transported per second at saturating concentration of the compound. Km is the concentration of the compound at which the compound is transported at half of Vmax. In general, a high value of Vmax is desirable for a substrate of a transporter. A low value of Km is desirable for transport of low concentrations of a compound, and a high value of Km is desirable for transport of high concentrations of a compound. Vmax is affected both by the intrinsic turnover rate of a transporter (molecules/transporter protein) and transporter density in plasma membrane which depends on expression level. For these reasons, the intrinsic capacity of a compound to be transported by a particular transporter is usually expressed as the ratio Vmax of the compound/Vmax of a control compound known to be a substrate for the transporter.

“Sustained release” refers to release of a therapeutic or prophylactic amount of the drug or an active metabolite thereof over a period of time that is longer than a conventional formulation of the drug. For oral formulations, the term “sustained release” typically means release of the drug within the GI tract lumen over a period of from about 2 to about 30 hours, more typically over a period of about 4 to about 24 hours. Sustained release formulations achieve therapeutically effective concentrations of the drug in the systemic blood circulation over a prolonged period of time relative to that achieved by oral administration of a conventional formulation of the drug. “Delayed release” refers to release of the drug or an active metabolite thereof into the gastrointestinal lumen after a delay time period, typically a delay of about 1 to about 12 hours, relative to that achieved by oral administration of a conventional formulation of the drug.

The phrases “specifically binds” when referring to a protein or “specifically immunoreactive with” when referring to an antibody, refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule such as antibody that specifically binds to a protein often has an association constant of at least 10⁵ M⁻¹, 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. However, some substrates of MCT transporters, have much lower affinities of the order of 10-10³ M⁻¹ and yet the binding can still be shown to be specific. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope hereof, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring.matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### DETAILED DESCRIPTION

I. General

MCT1 and MCT4 have been found to be consistently expressed at higher levels in many cancers than comparable normal cells of the same tissue. The present inventor has used this finding to isolate agents having useful pharmacological or imaging activity for treating or diagnosing cancer. In one embodiment, methods of identifying agents that inhibit MCT1 and/or MCT4 transporter activity are provided. Inhibition of such transporters expressed by cancer cells causes an increase in intracellular acidification of these cells. The increased acidification by itself or in combination with other chemotherapeutic agents kills or inhibits growth of the cancerous cells. In a second embodiment, methods of identifying agents, conjugates or conjugate moieties that are substrates for MCT1 and/or MCT4 are provided. Such agents, conjugates or conjugate moieties either have inherent cytotoxic activity or are linked to a cytotoxic moiety after screening to identify substrate activity. Administration of agents conjugates or conjugate moieties having inherent cytotoxic activity or linked to a component having such activity are preferentially accumulated in cancerous cells expressing MCT1 and/or MCT4 transporters. The cytotoxic activity by itself or in combination with another chemotherapeutic agent or radiation kills or inhibits growth of the cancerous cells. An analogous approach is used for imaging cancer cells except that the cytotoxic component is replaced by an imaging component. Agents, conjugates, or conjugate moieties having an inherent imaging component or linked to such a component are preferentially taken up by cancer cells overexpressing the MCT1 and/or MCT4 transporters. These cells can then be detected by using appropriate imaging methods such as positron emission technology, magnetic resonance imaging, or computed tomagraphy.

II. MCT1 and MCT4

The MCT family of transporters contains at least 14 members in humans. MCT transporters have 12 putative transmembrane domains, with both the amino and carboxy termini on the cytoplasmic side. Several members of the MCT family (MCT1, 2, 4) have been demonstrated to transport monocarboxylate molecules. Each of these have been shown to recognize a diverse group of small monocarboxylate metabolites such as lactate, pyruvate, butyrate, beta-hydroxybutyrate and nicotinate. Of the characterized MCT transporters, each has been shown to catalyze the net transport of one proton and one monocarboxylate. Transport is bidirectional, allowing transport either into or out of the cell depending on the substrate gradients. Because there is no net charge movement, transport does not depend on the membrane potential.

MCT transporters are highly dependent on pH. Transport rates are increased over 10-fold by lowering the pH one unit from 7.4 to 6.4. This strong dependence on pH provides the basis for MCT participation in pH regulation. As intracellular pH falls, efflux rates of lactate and protons increase, thereby counteracting the falling pH. In cells that undergo high rates of anaerobic glycolysis, such as skeletal muscle during strenuous exercise, the high rate of lactate production causes intracellular acidification and activates MCT lactic acid efflux. MCT1 and MCT4 are primarily responsible for mediating lactic acid efflux. MCT1 is more ubiquitous and has a higher affinity for lactate (˜2 mM), and is thought to mediate routine lactate efflux in most normal tissues. MCT4 has a lower affinity (˜40 mM), and exhibits a more restricted expression pattern. MCT4 is primarily found in skeletal muscle and other tissues that undergo anaerobic glycolysis. The low affinity of MCT4 and high turnover rate make it suitable for lactate efflux in cells that are capable of high rates of lactate production.

Energy metabolism in tumors differs significantly from the majority of normal tissues. Glucose is metabolized fully in well-oxygenated tissues to produce ATP, water, and CO₂. Tumor tissues are often poorly oxygenated (hypoxic), and inefficiently metabolize glucose via anaerobic glycolysis. As a result, tumor cells generate large amounts of lactic acid, which must be actively secreted from the cell to avoid a build-up of intracellular lactic acid and an acidification of the cytoplasm. High rates of lactic acid excretion combined with poor vascularization of solid tumors results in an acidic extracellular microenvironment. For most types of solid tumors, the extracellular pH has been estimated to range from pH 6.5-7.0, considerably lower than plasma pH (7.4). Secretion of lactic acid is efficient and tumor cells are able to maintain intracellular pH at normal levels (˜7.4). Tumor cells, like most normal cells, are unable to survive with an acid intracellular pH. Therefore, blocking pH regulation in tumors is likely to kill the tumors.

It is shown herein that MCT1 and MCT4 are strongly over-expressed in a high percentage of solid tumors. GenBank accession numbers for the human transporters are NM-003051 and NM-004207 respectively. Unless otherwise apparent from the context, reference to a transporter includes the amino acid sequence described in or encoded by the GenBank reference, and, allelic, cognate and induced variants and fragments thereof retaining essentially the same transporter activity. Usually such variants show at least 90% sequence identity to the exemplary Genbank nucleic acid or amino acid sequence.

III. Methods of Screening to Identify Substrates

Agents known or suspected to comprise a cytotoxic or imaging component can be screened directly for their capacity to act as substrates of MCT1 and/or MCT4. Alternatively, conjugate moieties can be screened as substrates, and the conjugate moieties linked to cytotoxic or imaging components. In such methods, the conjugate moieties can optionally be linked to a cytotoxic or imaging component, or other molecule during the screening process. If another molecule is used, the molecule is sometimes chosen to resemble the structure of a cytotoxic or imaging component ultimately intended to be linked to the conjugate moiety for pharmaceutical use. Alternatively, a conjugate moiety can be screened for a substrate activity alone and linked to a cytotoxic or imaging component after screening.

In some screening methods, the cells are transfected with DNA encoding a transporter. Oocytes and CHO (Chinese hamster ovary) cells, for example, are suitable for transfection. In other methods, natural cells expressing a transporter are used. Human embryonic kidney cells (HEKs), for example, naturally express the MCT1 transporter. In some methods, the cells only express MCT1 and/or MCT4. In 3other methods, cells express MCT1 and/or MCT4 in combination with other transporters. In still other methods, agents, conjugate moieties or conjugates are screened on different cells expressing different transporters. Agents, conjugate moieties or conjugates can be screened either for specificity for MCT1 or MCT4 or both.

Internalization of a compound evidencing passage through transporters can be detected by detecting a signal from within a cell from any of a variety of reporters. The reporter can be as simple as a label such as a fluorophore, a chromophore, or a radioisotope, Confocal imaging can also be used to detect internalization of a label as it provides sufficient spatial resolution to distinguish between fluorescence on a cell surface and fluorescence within a cell; alternatively, confocal imaging can be used to track the movement of compounds over time. In another approach, internalization of a compound is detected using a reporter that is a substrate for an enzyme expressed within a cell. Once the complex is internalized, the substrate is metabolized by the enzyme and generates an optical signal or radioactive decay that is indicative of uptake. Light emission can be monitored by commercial PMT-based instruments or by CCD-based imaging systems. In addition, assay methods utilizing LC/MS detection of the transported compounds or electrophysiological signals indicative of transport activity are also employed.

A preferred assay method determines whether an agent, conjugate or conjugate moiety is a substrate for MCT1 and/or MCT4 in cells expressing both MCT1 and/or MCT4 and an ion channel whose activity decreases with intracellular acidification (i.e., decreased intracellular pH). Because uptake of an agent, conjugate or conjugate moiety via MCT1 and/or MCT4 involves co-uptake of a proton, the uptake results in intracellular acidification or decreased intracellular pH. The decrease in pH inhibits activity of the ion channel reducing ion current across the plasma membrane. The ion current can be measured by voltage clamping cells and determining the current required to hold the voltage constant (as described in PCT/US02/18686 incorporated by reference). The potassium ion channel ROMK (Genbank accession number NM_{—}000220; see also Bock et al., Gene 188(1), 9-16 (1997)) is suitable for use in these methods. Usually, the ion channel, the co-transporter(s) or both are expressed from a nucleic acid transformed into the cell. The nucleic acids can be DNA or mRNA. The activity of the ion channel is reduced to undetectable levels on decreasing intracellular pH to 6.5. However, the activity is essentially independent of extracellular pH.

The same principles can also be used more generally for determining intracellular pH levels. In this case, one simply determines the ion current across the plasma membrane of a cell expressing a pH-sensitive ion transporter. One then determines a measure of the intracellular pH from a relationship between intracellular pH and current. That is, current decreases with a decrease in intracellular pH below physiological, or in other words current decreases with an increase in intracellular acidification.

The same principles can also be used to monitor flux of an organic solute across a cell. In this case, a cell expressing a pH-sensitive ion channel and a co-transporter of the organic solute and a proton is contacted with the organic solute. Flux of the solute is monitored by monitoring the ion current across the plasma membrane. The flux of the solute is inversely related to the magnitude of the ion current. Thus, a measure of the flux of the solute can be determined from the magnitude of the current.

In some methods, multiple agents, conjugate moieties or conjugate moieties are screened simultaneously and the identity of each agent, conjugate or conjugate moiety is tracked using tags linked to the agents or conjugate moieties. In some methods, a preliminary step is performed to determine binding of an agent, conjugate or conjugate moiety to a transporter. Although not all agents, conjugates or conjugate moieties that bind to a transporter are substrates of the transporter, observation of binding is an indication that allows one to reduce the number of candidate substrates from an initial repertoire. In some methods, the transport rate of an agent, conjugate or conjugate moiety is tested in comparison with the transport rate of a reference substrate for that transporter. For example, lactate, a natural substrate of MCT1 and MCT4 can be used as a reference. The comparison can either be performed in separate parallel assays in which an agent, conjugate or conjugate moiety under test and the reference substrate are compared for uptake on separate samples of the same cells. Alternatively, the comparison can be performed in a competition format in which an agent, conjugate or conjugate moiety under test and the reference substrate are applied to the same cells. Typically, the agent, conjugate or conjugate moiety and the reference substrate are differentially labeled in such assays.

In such comparative assays, the Vmax of an agent, conjugate or conjugate moiety, tested can be compared with that of the reference substrate. If an agent, conjugate moiety or conjugate has a Vmax of at least 1%, 5%, 10%, 20%, and most preferably at least 50% of the reference substrate for the transporter then the agent, conjugate moiety or conjugate can be considered to be a substrate for the transporter. In general, the higher the Vmax of the agent, conjugate moiety or conjugate relative to that of the reference substrate the better. Therefore, agents, conjugate moieties or conjugates having Vmax's of at least 1%, 10%, 20%, 50%, 100%, 150% or 200% (i.e., two-fold) of the Vmax of the reference substrate for the transporter are screened in some methods. The agents to which conjugate moieties are linked can by themselves show little or no detectable substrate activity for the transporter (e.g., Vmax relative to that of a reference substrate of less than 0.1% or 1%).

Having determined that an agent, conjugate or conjugate moiety is a substrate for MCT1 and/or MCT4, a further screen can be performed to determine cytotoxic activity against cancerous cells. If the agent, conjugate or conjugate moiety does not have inherent cytotoxic activity it is first linked to a cytotoxic component. The agent, conjugate or conjugate moiety is then contacted with a cancerous cell expressing MCT1 and/or MCT4. The contacting can be performed either on a population of cancerous cells in vitro, or in a cancer tissue in an animal. Optionally, the animal can be a tumor xenograft model. Cytotoxic activity of the agent, conjugate or conjugate moiety is determined from an effect of killing or inhibiting growth of the cancerous cells. Optionally, the effect of the agent, conjugate or conjugate moiety can be compared with a placebo. Cytotoxicity assays are preferably performed on lung cancer, colon cancer, breast cancer or prostate cancer cells, and or brain cancer cells, or combinations thereof.

A further screen can be performed to determine toxicity of the agent, conjugate, or conjugate moiety to normal cells. The agent, conjugate or conjugate moiety, either inherently having a cytotoxic component or linked to a cytotoxic component, is administered to a laboratory animal. Various tissues of the animal, such as liver, kidney, heart and brain are then examined for signs of pathology.

An additional screen can be performed to check that agents, conjugates or conjugate moieties substantially capacity for passive diffusion into cells. Such an assay can be performed using the same approach as the substrate assay except that the cells lack MCT1 and MCT4 transporters. In such assays, little or no passive diffusion of agents, conjugates or conjugate moieties into cells is desired.

IV. Methods of Screening to Identify Inhibitors

Agents are usually initially screened for inhibitory activity using an assay to determine specific binding to an MCT1 and/or MCT4 transporter. The assay is usually performed with the MCT1 and/or MCT4 transporter expressed from cells. This format identifies ligands that bind to the extracellular domain of MCT1 and/or MCT4 transporter. These ligands may or may not have inhibitory activity. Agents can be prescreened to eliminate those that bind specifically or otherwise to control cells lacking MCT1 and MCT4 transporters.

Ligands that specifically bind to an MCT1 and/or MCT4 transporter are then further screened for inhibitor activity using a cell uptake assay. Such an assay is performed essentially as described in Section III except that the agent is screened in competition with a known substrate of MCT1 and/or MCT4 and the desired activity is a capacity of the agent to inhibit uptake of the known substrate. A preferred format uses cells expressing a pH-sensitive ion channel and MCT1 and/or MCT4 and a known substrate of MCT1 and/or MCT4. Inhibitor activity of an agent is shown by increased intracellular currents relative to a control assay in which the agent is absent. Preferably, an agent with inhibitory activity reduces the Vmax and/or increases the Km of a known substrate such as lactate for MCT1 and/or MCT4 by at least 1, 5, 10, 50, 100, 500 or 1000%. Noncompetitive inhibitors reduce Vmax, competitive inhibitors increase Km.

Further screens to determine cytotoxic activity, side-effects in an undiseased animal and lack of passive diffusion can be performed for inhibitors in similar fashion to those described for substrates in Section III. Preferably cytotoxicity screens are performed on a cancer other than a melanoma. If an inhibitor inhibits MCT1, the cytotoxicity screen is preferably performed on a cancer other than a melanoma or brain cancer. Cytotoxicity assays are preferably performed on lung cancer, colon cancer, breast cancer or prostate cancer cells, and in the case of MCT4 inhibitors, brain cancer cells.

V. Agents, Cytotoxic Agents, Imaging Components

The agents, conjugate or conjugate moieties to be screened as substrates of MCT1 and/or MCT4 are usually monocarboxylate compounds. Agents to be screened as inhibitors of MCT1 and/or MCT4 can also be monocarboxylates or analogs thereof. However, other agents that are not structurally related to natural substrates of monocarboxylates can also be screened as inhibitors. Agents can be obtained from natural sources such as, e.g., marine microorganisms, algae, plants, and fungi. Alternatively, agents can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, heterocyclic and other organic compounds, lipids, glucocorticoids, antibiotics, peptides, sugars, carbohydrates, and chimeric molecules.

Typically if an agent is being screened, the agent is known or suspected to have an inherent cytotoxic or imaging component. If a conjugate is being screened, the conjugate usually comprises an agent being screened for substrate activity linked to a known cytotoxic or imaging component. If a conjugate moiety is being screened, the conjugate moiety typically lacks a cytotoxic or imaging component and this is added after screening.

Suitable cytotoxic components for incorporation into conjugates or linkage to conjugate moieties after screening include platinum, nitrosourea, nitrogen mustard, a phosphoramide group that is only cytotoxic to cancer cells when taken up active transport. Radiosensitizers, such as nitroimidizoles, can also be used. The choice of imaging component depends on the means of detection. For example, a fluorescent imaging component is suitable for optical detection. A paramagnetic imaging component is suitable for tomographic detection without surgical intervention. Radioactive labels can also be detected using PET or SPECT.

The agents, conjugates or conjugate moieties to be screened optionally linked to a cytotoxic or imaging component if not inherently present are preferably small molecules having molecular weights of less than 1000 Da and preferably less than 500 Da.

VI. Linkage of Cytotoxic or Imaging Components to Substrates

Conjugates can be prepared by either by direct conjugation of a cytotoxic or imaging component to a substrate for MCT1 and/or MCT4 with a covalent bond (optionally cleavable in vivo), or by covalently coupling a difunctionalized linker precursor with the cytotoxic or imaging component and substrate. The linker precursor is selected to contain at least one reactive functionality that is complementary to at least one reactive functionality on the cytotoxic or imaging component and at least one reactive functionality on the substrate. Optionally, the linker is cleavable. Suitable complementary reactive groups are well known in the art as illustrated below:

### Complementary Binding Chemistries

| | Second | |
|---|---|---|
| First Reactive Group | Reactive Group | Linkage |
| hydroxyl | carboxylic acid | ester |
| hydroxyl | haloformate | carbonate |
| thiol | carboxylic acid | thioester |
| thiol | haloformate | thiocarbonate |
| amine | carboxylic acid | amide |
| hydroxyl | isocyanate | carbamate |
| amine | haloformate | carbamate |
| amine | isocyanate | urea |
| carboxylic acid | carboxylic acid | anhydride |
| hydroxyl | phosphorus acid | phosphonate or phosphate |
| | | ester |

VII. Pharmaceutical Compositions

The above screening processes result several entities to be incorporated into pharmaceutical compositions. These entities include agents that are both substrates for MCT1 and MCT4 and have an inherent cytotoxic or imaging component. The entities also include conjugates in which a cytotoxic or imaging component is linked to a substrate for MCT1 or MCT4. The entities also include inhibitors of MCT1 and/or MCT4.

The above entities are combined with pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS), Ringer's solution, dextrose solution, and Hank's solution. In addition, the pharmaceutical composition or formulation can also include other carriers, adjuvants, or nontoxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like (see, e.g., Remington's Pharmaceutical Sciences, Mace Publishing Company, Philadelphia, Pa., 17th ed. (1985); for a brief review of methods for drug delivery, see, Langer, Science 249:1527-1533 (1990); each of these references is incorporated by reference in its entirety).

Pharmaceutical composition can be administered topically, orally, intranasally, intradermally, subcutaneously, intrathecally, intramuscularly, topically, intravenously, or injected directly to a site of cancerous tissue. For parenteral administration, the compounds disclosed herein can be administered as injectable dosages of a solution or suspension of the compound in a physiologically acceptable diluent with a pharmaceutical carrier which can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymers thereof for enhanced adjuvant effect, as discussed above (see Langer, Science 249, 1527 (1990) and Hanes, Advanced Drug Delivery Reviews 28, 97-119 (1997). The pharmaceutical compositions disclosed herein can be administered in the form of a depot injection or implant preparation which can be formulated in such a manner as to permit a sustained or pulsatile release of the active ingredient.

Pharmaceutical compositions for oral administration can be in the form of e.g., tablets, pills, powders, lozenges, sachets, cachets, elixirs, suspensions, emulsions, solutions, or syrups. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. Preserving agents such as methyl- and propylhydroxy-benzoates; sweetening agents; and flavoring agents can also be included. Depending on the formulation, compositions can provide quick, sustained or delayed release of the active ingredient after administration to the patient. Polymeric materials can be used for oral sustained release delivery (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228:190; During et al., 1989, Ann. Neurol. 25:351; Howard et al, 1989, J. Neurosurg. 71:105). Sustained release can be achieved by encapsulating conjugates within a capsule, or within slow-dissolving polymers. Preferred polymers include sodium carboxymethylcellulose, hydroxypropylcellulose, hydroxypropylmethylcellulose and hydroxyethylcellulose (most preferred, hydroxypropylmethylcellulose). Other preferred cellulose ethers have been described (Alderman, Int. J. Pharm. Tech. & Prod. Mfr., 1984, 5(3) 1-9). Factors affecting drug release have been described in the art (Bamba et al., Int. J. Pharm., 1979, 2, 307). For administration by inhalation, the compounds for use according to the disclosures herein are conveniently delivered in the form of an aerosol spray preparation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Effective dosage amounts and regimes (amount and frequency of administration) of the pharmaceutical compositions are readily determined according to any one of several well-established protocols. For example, animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The components of pharmaceutical compositions are preferably of high purity and are substantially free of potentially harmful contaminants (e.g., at least National Food (NF) grade, generally at least analytical grade, and more typically at least pharmaceutical grade).

To the extent that a given compound must be synthesized prior to use, the resulting product is typically substantially free of any potentially toxic agents, particularly any endotoxins, which may be present during the synthesis or purification process. Compositions are usually made under GMP conditions. Compositions for parenteral administration are usually sterile and substantially isotonic.

VIII. Methods of Treatment

The pharmaceutical compositions disclosed herein are used in methods of treating or preventing cancer. Examples of tumors amenable to treatment are cancers of the bladder, brain, breast, colon, esophagus, kidney, leukemia, liver, lung, oral cavity, ovary, pancreas, prostate, skin, stomach and uterus. The compositions are particularly useful for treating solid tumors, such as sarcoma, lymphomas and carcinomas. If a pharmaceutical composition comprises an entity which is a substrate or inhibitor of MCT1, then optionally the cancer is not a brain cancer or a melanoma. If a pharmaceutical composition comprises an entity which is a substrate or inhibitor of MCT4, then optionally the cancer is not a melanoma. Preferred cancers for treatment are those shown in Table 1 in which expression of MCT1 and/or MCT4 is higher in the cancer than in normal cells from the tissue. Examples of these cancers include brain cancers, such as astrocytoma, glioblastoma multiforme, malignant ependymana, and medullablastoma. Breast cancers amenable to treatment include infiltrating ductal adenocarcinoma, ductal adenocarcinoma, and lobular adenocarcinoma. Lung cancers amenable to treatment include squamous cell carcinoma and epidermoid carcinoma. Colon cancers amenable to treatment include colon adenocarcinoma, medullary carcinoma, and mucinous carcinoma. Prostate cancers amenable to treatment include prostate sarcoma. Incorporation of other isotopes such as boron (¹⁰B) allows boron neutron capture therapies (BNCT) in which low-energy neutron irradiation is used to induce boron decay and release of higher energy particles that are toxic to cells. An advantage this and similar approaches relative to existing chemotherapy approaches is that release of particles from decaying isotopes could kill neighboring cells as well, and provide more complete tumor killing in poorly vascularized solid tumors. Another advantage of these approaches is that tumors in highly radiation sensitive tissues (liver, pancreas) can be targeted.

In prophylactic applications, pharmaceutical compositions are administered to a patient susceptible to, or otherwise at risk of, cancer in an amount and frequency sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, pharmaceutical compositions are administered to a patient suspected of, or already suffering from such a disease in an amount and frequency sufficient to cure, or at least partially arrest, the symptoms of the disease (biochemical, histologic and/or behavioral), including its complications and intermediate pathological phenotypes in development of the disease. An amount of pharmaceutical composition sufficient to achieve at least one of the above objects is referred to as an effective amount, and a combination of amount and frequency sufficient to achieve at least one of the above objects is referred to as an effective regime.

Optionally, administration of a pharmaceutical composition is combined with administration of a second chemotherapeutic agent or radiation. For example, in some methods, the pharmaceutical composition comprises a substrate of MCT1 and/or MCT4 linked to a cytotoxic component that renders a cell susceptible to radiation damage. Alternatively, if the pharmaceutical composition comprises an agent that inhibits MCT1 and/or MCT4 transport to increase the intracellular pH of cancer cells, then administration of the pharmaceutical composition can be combined with administration of a chemotherapeutic agent that is particular toxic under conditions of low intracellular pH. Several chemotherapy drugs have been demonstrated to have greater activity in cells with a more acidic cytoplasm. One compound, lonidamide, readily acidifies the cytoplasm, and has shown promise in Phase 3 clinical trials for enhancing the activity of several current chemotherapy regimens. Lonidamide has a complex mechanism of action, which includes inhibition of glycolytic metabolism and inhibition of lactate transport. In addition, lonidamide is a lipophilic weak acid that can acidify cells directly by passive diffusion. A sulfur-containing platinum compound (thioplatin) is more cytotoxic at low pH and has shown efficacy in mouse xenograft models, with lower toxicities than cisplatin. In addition, new camptothecin analogs have been developed that are highly pH sensitive. Both of these classes of compounds are known to have activity against a broad range of tumor types, and exhibit some intrinsic specificity towards tumor cells. However, they also exhibit toxicities that can limit their clinical utility absent combination with MCT1 and/or MCT4 inhibition in the present methods. Combination of pH-sensitive versions of these compounds with pharmaceutical compositions that inhibit MCT1 and/or MCT4 provides a highly selective tumor therapy with reduced side effects. Optionally, administration of an inhibitor of MCT4 can be combined with an inhibitor of MCT1.

IX. Methods of Imaging

As discussed above, conjugates are provided comprising a substrate of MCT1 and/or MCT4 linked to an imaging component, and agents that are both substrates for MCT1 and/or MCT4 and have an inherent imaging component, and pharmaceutical compositions comprising either of these entities. These pharmaceutical compositions can be used for in vivo imaging. The compositions are administered to a patient and preferentially taken up by cancer cells expressing MCT1 and/or MCT4 in the patient. The imaging component is then detected. In some method, the imaging component is also a cytotoxic component. For example many radioisotopes are suitable for both imaging and cytotoxic activity. In such cases, methods of imaging and methods of treatment can be combined. Currently used diagnostic imaging techniques include positron emission tomography (PET), magnetic resonance imaging (MRI), and computed tomography (CT). Actively transported imaging components provide information about the presence of a tumor, and the extent of MCT1 and/or MCT4 transporter activity in the tumor. Knowledge of abundant MCT1 and/or MCT4 transporter activity has diagnostic activity in indicating that treatment using the methods described herein is likely to be successful.

X. Methods of Grading

Provided herein are methods of grading a cancer by determining expression levels of MCT1 and/or MCT4 transporters. Expression levels can be determined by measuring mRNA or protein levels. mRNA levels can be measured using microarrays or quantitative PCR for example. Protein levels can be measured using immunoassays such as a Western blot. Expression levels in cancerous cells from a patient are compared with expression levels of control cells. The control cells are usually healthy non-cancerous cells from the same tissue, preferably from the same patient, as the cancerous cells. Increased expression of MCT1 and/or MCT4 in cancerous cells relative to control cells signal that the cancerous cells are amenable to treatment by the methods disclosed herein. Preferably grading is performed on a cancer other than a melanoma. If grading is based on expression of MCT1 and not MCT4, the cytotoxicity screen is preferably performed on a cancer other than a melanoma or brain cancer. Cytotoxicity assays are preferably performed on lung cancer, colon cancer, breast cancer or prostate cancer cells, and in the case of MCT4 inhibitors, brain cancer cells. Optionally, analysis of expression levels of MCT1 and/or MCT4 transporters can be combined with other transporters that may be preferentially expressed in cancerous cells and enzymes that influence tumor sensitivity or resistance to drugs.

### EXAMPLES

Identification of Transporter Genes Strongly Expressed in Common Solid Tumors.

Fourteen transporters belonging to the MCT family were identified using translated BLAST searches of the fully sequenced human genome. Because the BLAST searches were performed on the raw human genome, it is likely that the fourteen family members is a complete list.

Quantitative PCR primers were developed and validated against each of the fourteen MCT family members. To gain preliminary information about MCT expression in human tumors, approximately 60 tumor RNA samples were purchased from a number of commercial vendors (Ambion, Inc. of Austin Tex.; BD Bioscience/Clontech, Palo Alto, Calif.; and Ardais Corp., Lexington, Mass.) representing a wide range of solid tumors and some normal organ tissue, and in some cases normal and tumor tissue from the same patient. Single-stranded cDNA was generated from each tissue and all fourteen MCT transporters were tested for expression in tumor samples using SYBR green real-time PCR. To compare between tumor samples, three ubiquitous genes (GAPDH, 23kDA, and HPRT) were used to normalize each sample. Tumor samples appear to express these genes at levels similar to normal tissues and cell lines. MCT1 and MCT4 are highly expressed in solid tumors. Nearly every tumor sample exhibited high levels of MCT1 or MCT4 expression. MCT1 or MCT4 levels exceeded 30% of GLUT1, a known tumor transporter, in most tumor samples. Expression of other MCT transporters is detectable, but their mRNA levels are typically less than 10% of either MCT1 or MCT4.

Our PCR findings confirm that several MCTs are well expressed in tumor samples, MCT1 and MCT4 being most strongly expressed. However, these qPCR studies do not readily establish whether these transporters are truly overexpressed compared with normal tissues. Normal tissue RNA is not as widely available as that from tumor tissues. In addition RNA is typically extracted from complex tissues with many cell types, which makes it difficult to determine expression levels in specific cell types. Furthermore, PCR analysis only provides information about mRNA expression, which can often differ significantly from protein expression. To overcome these problems, antibody staining of tissue slide arrays was used.

Characterization of MCT1 and MCT4 Expression in Tumor Tissue Arrays

Tumor tissue arrays were purchased from Ambion, Inc., Austin, Tex. and Biogenix/InnoGenex, San Ramon, Calif. These arrays typically consist of 10-500 formalin-fixed paraffin-embedded tumor samples arrayed on a single microscopy slide. The tumor arrays were derived from lung, colon, prostate, breast, and brain tumors. In addition, matched normal or benign samples were present on most of the arrays. Such arrays allow the rapid determination of protein levels in a large number of tumor or normal tissue samples. To develop antibodies against MCT1 and MCT4, we synthesized two different GST-fusion (glutathion-S-transferase-fusion) proteins using peptides from the C-terminus of MCT1 and MCT4, respectively. The first GST-fusion protein was comprised of the glutathion-S-transferase protein bound to a 55 amino acid chain portion of the MCT1 transporter (the 55 amino acids from the C-terminus of MCT1) and is designated GST-MCT1 in Table 1. The second GST-fusion protein was comprised of the glutathion-S-transferase protein bound to a 58 amino acid chain portion of the MCT4 transporter (the 58 amino acids from the C-terminus of MCT4) and is designated GST-MCT4 in Table 1. The purified GST-fusion proteins were each injected in rabbits. Specific antibodies were affinity purified from rabbit sera using a column coated with the fusion protein.

We next proceeded to stain over 1000 tumor samples. Staining intensity was scored on a scale of 0-2 and the results of the staining studies are summarized in Table 1. Both MCT proteins were well expressed in tumor biopsy samples.

Although MCT1 was observed in normal tissue, it is also clearly expressed at elevated levels in many of the tumor types. In particular, glioblastoma multiforme appears to exhibit unusually high levels of MCT1. MCT4 was not as well expressed in normal tissues, but was dramatically overexpressed in a high-percentage of tumor samples, especially in lung and colon tumors.

Using Ambion tumor tissue slide arrays cut from the same block of tissue cores, several different proteins in the same tumor samples were compared. When both MCT1 and MCT4 are tested against the same set of biopsy samples, nearly all tumors express one or both of these transporters. Therefore, few if any tumors completely lack MCT1 and/or MCT4 expression. In addition, staining for most samples appears to be rather uniform throughout the tumor sample. Comparison with normal tissue also indicates that MCT1 and MCT4 are upregulated in at least 50% of tumor samples, compared to non-tumor tissue taken from the same patient. These findings, summarized in Table 1, clearly demonstrate that MCT1 and MCT4 are highly expressed in common tumors.

TABLE 1

| | | Stain Score | | | | | Average | Percentage of |
|---|---|---|---|---|---|---|---|---|
| Antigen: | | 0 | 0.5 | 1 | 1.5 | 2 | Stain Score | Scores ≧ 1.5 |
| | Brain Cancer Type | | | | | | | |
| GST- | Astrocytoma | 0 | 2 | 6 | 0 | 1 | 1.0 | 11 |
| MCT1 | Glioblastoma multiforme | 0 | 7 | 13 | 7 | 7 | 1.2 | 41 |
| | Malignant ependymana | 0 | 0 | 0 | 2 | 0 | 1.5 | 100 |
| | Medullablastoma | 0 | 0 | 4 | 1 | 1 | 1.3 | 33 |
| | Normal brain (control) | 0 | 1 | 7 | 0 | 0 | 1.0 | 0 |
| GST- | Astrocytoma | 0 | 1 | 7 | 1 | 0 | 1.0 | 11 |
| MCT4 | Glioblastoma multiforme | 0 | 8 | 14 | 12 | 0 | 1.1 | 35 |
| | Malignant ependymana | 0 | 0 | 0 | 2 | 0 | 1.5 | 100 |
| | Medullablastoma | 0 | 2 | 3 | 0 | 1 | 1.0 | 17 |
| | Normal brain (control) | 0 | 6 | 2 | 0 | 0 | 0.6 | 0 |
| | Breast Cancer Type | | | | | | | |
| GST- | Infiltrating Ductal | 3 | 21 | 10 | 2 | 2 | 0.7 | 11 |
| MCT1 | Adenocarcinoma | | | | | | | |
| | Ductal Adenocarcinoma | 0 | 3 | 0 | 1 | 1 | 1.0 | 40 |
| | Lobular adenocarcinoma | 0 | 0 | 2 | 0 | 0 | 1.0 | 0 |
| | Mucinous carcinoma | 0 | 1 | 0 | 0 | 0 | 0.5 | 0 |
| | Normal breast (control) | 2 | 7 | 0 | 0 | 0 | 0.4 | 0 |
| GST- | Infiltrating Ductal | 1 | 11 | 15 | 9 | 2 | 1.0 | 29 |
| MCT4 | Adenocarcinoma | | | | | | | |
| | Ductal Adenocarcinoma | 0 | 1 | 3 | 1 | 0 | 1.0 | 20 |
| | Lobular adenocarcinoma | 0 | 1 | 1 | 0 | 0 | 0.8 | 0 |
| | Mucinous carcinoma | 0 | 1 | 0 | 0 | 0 | 0.5 | 0 |
| | Normal breast (control) | 1 | 6 | 2 | 0 | 0 | 0.6 | 0 |
| | Lung Cancer Type | | | | | | | |
| GST- | Adenocarcinoma | 0 | 14 | 8 | 0 | 0 | 0.7 | 0 |
| MCT1 | Squamous Cell | 0 | 1 | 5 | 4 | 4 | 1.4 | 57 |
| | Carcinoma | | | | | | | |
| | Epidermoid Carcinoma | 0 | 1 | 1 | 0 | 0 | 0.8 | 0 |
| | Malignant Mesothelioma | 0 | 0 | 1 | 0 | 0 | 1.0 | 0 |
| | Normal lung (control) | 0 | 12 | 6 | 0 | 0 | 0.7 | 0 |
| GST- | Adenocarcinoma | 0 | 1 | 5 | 7 | 7 | 1.5 | 70 |
| MCT4 | Squamous Cell | 0 | 0 | 5 | 5 | 3 | 1.4 | 61 |
| | Carcinoma | | | | | | | |
| | Epidermoid Carcinoma | 0 | 0 | 1 | 1 | 0 | 1.3 | 50 |
| | Malignant Mesothelioma | 0 | 0 | 1 | 0 | 0 | 1.0 | 0 |
| | Normal lung (control) | 0 | 6 | 7 | 3 | 1 | 1.0 | 23 |
| | Colon Cancer Type | | | | | | | |
| GST- | Colon Adenocarcinoma | 0 | 5 | 11 | 15 | 12 | 1.4 | 63 |
| MCT1 | Medullary Carcinoma | 0 | 0 | 0 | 2 | 0 | 1.5 | 100 |
| | Mucinous Carcinoma | 0 | 2 | 0 | 1 | 0 | 0.8 | 33 |
| | Normal colon (control) | 1 | 2 | 5 | 0 | 1 | 0.9 | 11 |
| GST- | Colon Adenocarcinoma | 0 | 2 | 15 | 16 | 12 | 1.4 | 62 |
| MCT4 | Medullary Carcinoma | 0 | 0 | 1 | 0 | 2 | 1.7 | 67 |
| | Mucinous Carcinoma | 0 | 1 | 1 | 1 | 0 | 1.0 | 33 |
| | Normal colon (control) | 0 | 3 | 3 | 2 | 1 | 1.1 | 33 |
| | Prostate Cancer Type | | | | | | | |
| GST- | Adenocarcinoma | 3 | 10 | 8 | 5 | 5 | 1.0 | 32 |
| MCT1 | Prostate sarcoma | 0 | 0 | 0 | 0 | 1 | 2.0 | 100 |
| | Normal Prostate (control) | 1 | 2 | 5 | 7 | 6 | 1.4 | 62 |
| GST- | Adenocarcinoma | 9 | 18 | 3 | 0 | 0 | 0.4 | 0 |
| MCT4 | Prostate sarcoma | 0 | 0 | 1 | 0 | 0 | 1.0 | 0 |
| | Normal Prostate (control) | 10 | 11 | 1 | 0 | 0 | 0.3 | 0 |

Studies of Cloned MCT Transporters

To assess transport function of a specific transporter protein, it is preferable to clone the cDNA and express the protein in cells that have low endogenous transport activity. Human MCT1 and MCT4 were cloned by PCR and fully sequenced. In addition CD147, a reported cofactor for MCT transporters, was cloned. Both MCT1 and MCT4 were subcloned into plasmids that can be used for expression in mammalian cells or Xenopus oocytes. Because most cell lines already exhibit high levels of MCT transport, much of our characterization has been performed in Xenopus oocytes which have low levels of endogenous MCT expression. For expression in Xenopus oocytes, in vitro cRNA was prepared and injected into defoliculated oocytes.

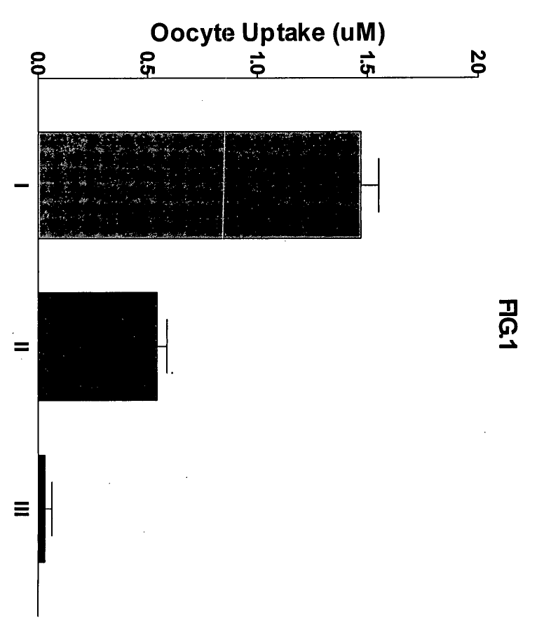

Oocytes expressing MCT1 or MCT4 RNA exhibited higher levels of ¹⁴C lactate uptake. Co-injection of CD147 did not lead to additional transport activity, and was not further evaluated. To measure directly the uptake of possible MCT substrates, an oocyte uptake assay in which compounds are measured by mass spectroscopy was developed. To illustrate this approach, uptake of 2-thiopheneglyoxylic acid (2-TPGA) is shown in FIG. 1. Oocytes used in this experiment were either injected with rMCT1 (I) or hMCT4 (II) RNA and incubated at 16-18° C. until maximal transporter expression was reached. Oocytes from the same batch, which were not injected with RNA, were used in the experiment to serve as a control (III). A 1 mM solution of 2-TPGA was prepared in oocyte ringers (ND96) buffer (90 mM NaCl, 10 mM HemiNa HEPES, 2 mM KCl, 1 mM MgCl₂, 1.8 mM CaCl₂), pH 7.2 containing 0.5% bovine serum albumin. The 2-TPGA was then administered to pools of 8 oocytes for a 4 min duration. Following the incubation, the pools of oocytes were washed 4 times with 0.5% BSA ND96 buffer and separated into 2 oocyte subpools containing 4 oocytes each. Subpools were homogenized in 150 μl of ice cold 80% MeOH/H₂O, and lysed manually with a P200 pipettor. Lysates were vortexed briefly before being spun in a 4° C. tabletop centrifuge at 13.2 krpm for 15 min. Approximately 110 μl of lysate was removed from the Eppendorf tubes and placed in a 96-well plate. Lysates were analyzed for 2-TPGA concentrations by liquid chromatography-mass spectroscopy.

A mammalian cell assay for MCT transport was also developed. Approximately one dozen human cell lines were screened for active pH-dependent lactate transport. From this screen, HEK cells were chosen for further examination. MCT1 was found by quantitative PCR to be the primary MCT expressed in HEK cells as shown in Table 2:

TABLE 2
Monocarboxylate Transporter Expression
in HEK Peak Cells

| | Number of RNA |
|---|---|
| Transporter | Transcripts |
| MCT 1 | 224,026 |
| MCT 9 | 21,640 |
| MCT7 | 10,855 |
| MCT11 #2 | 10,639 |
| MCT2 | 5,199 |
| MCT10 | 1,939 |
| MCT8 | 1,253 |
| MCT5 | 789 |
| MCT3 | 257 |
| MCT6 | 225 |
| MCT4 | 29 |

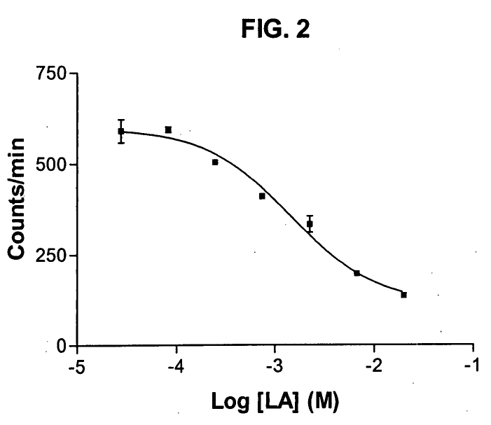

The data in Table 2 show that MCT1 RNA transcripts were more than 10-fold more abundant than the second-most abundant MCT in HEK Peak cells, making these cells an excellent choice for an MCT1 assay. To quickly screen for compounds that interact with MCT1, a radiolabeled lactate competition assay was developed. In this assay, MCT1 expressing HEK cells are exposed to a plurality of solutions containing the known radiolabeled lactate substrate and the test compound. Each of the plurality of solutions contains a set concentration of the radiolabeled lactate. Each of the plurality of solutions also contains a different concentration of the test compound. If the test compound is an active substrate for MCT transport, it will compete with the radiolabeled substrate, causing less of the radiolabeled substrate to be transported into the HEK cells. The amount of radiolabeled lactate which is taken up by the cells can be accurately measured by lysing the cells and measuring the radioactive counts per minute. A representative test compound curve generated using non-radiolabeled lactate as a model test compound is shown in FIG. 2. FIG. 2 is a plot of the total (i.e., total of both the radiolabeled and non-radiolabeled) lactate concentration ([LA]) versus the radioactive counts per minute measured in the cell lysate. Because the non-radiolabeled lactic acid competes with the radiolabeled lactate, the counts per minute becomes lower as the concentration of non-radiolabeled lactate is increased, forming the characteristic reverse-S-shaped dose response curve. For test compounds that are not substrates for the MCT1 transporter, the curve remains an essentially flat line (not shown in FIG. 2), i.e., there is no dose response seen. This assay was performed in 96-well plates with HEK cells adherent on the clear plastic bottoms of the wells. All addition and washing of solutions is automated. Based on our profiling data and uptake pharmacology, we conclude that HEK cells are a good model for MCT1 transport.

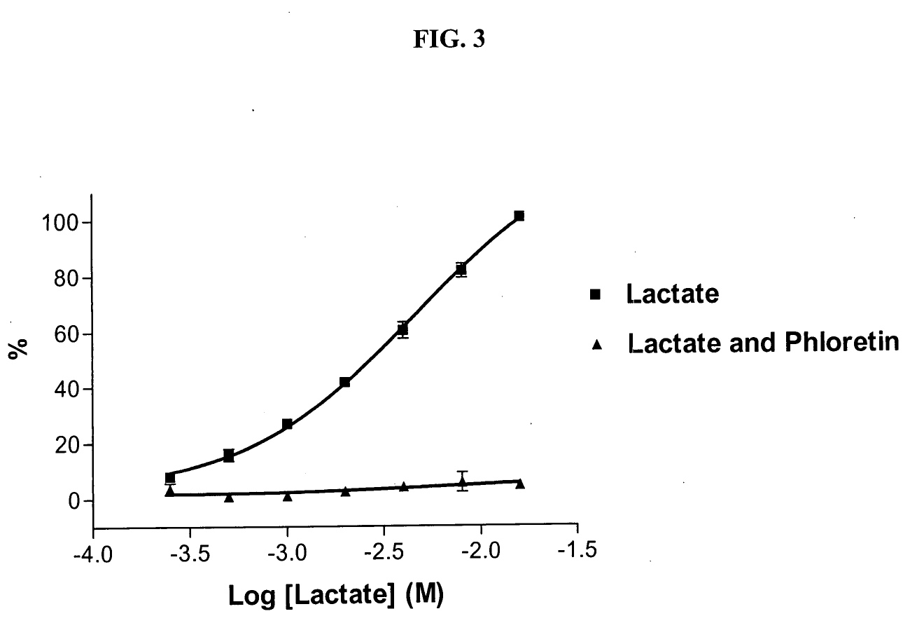

The competition uptake assay in HEKs only demonstrates that a molecule inhibits MCT1 transport, and does not demonstrate whether the molecule is a true substrate that is actively transported across the plasma membrane. Because MCT1 transports both the carboxylate anion and the corresponding proton, a pH measuring assay was developed to measure net transport of protons into the cytoplasm. Intracellular pH can be measured by a pH-sensitive fluorescent dye such as BCECF. When lactate is applied to HEK cells, there is a dose-dependent intracellular acidification. The compound phloretin, when applied to HEK cells by itself, does not cause an intracellular pH change. However, when phloretin is applied to HEK cells together with lactate, the phloretin completely blocks the lactate response. In the phloretin inhibition assay, the HEK cells are first exposed to a plurality of solutions containing various concentrations of the test compound (in FIG. 3, the model test compound is lactate) and then to the same solutions to which have been added a set (0.25 mM) concentration of phloretin. Thus, FIG. 3 is a graph of lactate concentration ([L]) versus the percent of maximum pH change found with administration of the highest concentration of the test compound (lactate) alone. The two curves shown in FIG. 3 show that when the cells are exposed to lactate with no phloretin present, the measured pH within the cells changes as the concentration of lactate in the external solution changes. This is because MCT1 co-transports lactate and a proton into the HEK cells, causing their internal pH to decrease. This pH lowering effect is shown by the characteristic reverse-S-shaped dose response upper curve in FIG. 3. When the cells are exposed to the solutions containing both lactate and phloretin, the phloretin inhibits the lactate uptake by MCT1 and there is little pH change in the cells, as shown by the relatively flat lower curve in FIG. 3. The phloretin inhibition assay can also be used to determine those carboxylic acids that are actively transported MCT substrates from those that are only passively absorbed across the cell membrane. In the latter case, when a passive absorbed acid is applied to HEK cells, there is an intracellular acidification, which occurs both with and without phloretin.

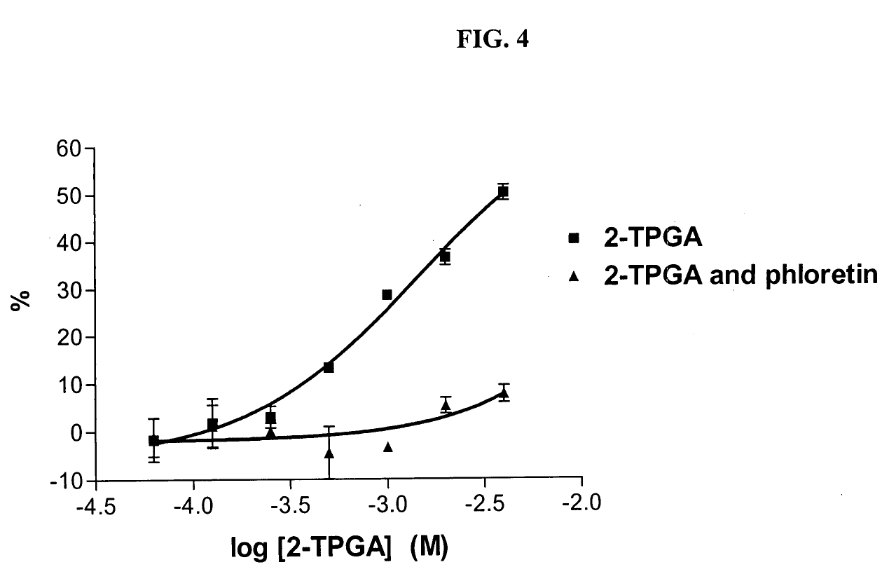

The phloretin inhibition assay results for 2-thiophene glyoxylic acid (2-TPGA) are shown in FIG. 4. FIG. 4 is a graph of 2-TPGA concentration ([2-TPGA]) versus the relative percent decrease in intracellular pH compared to a standard decrease in intracellular pH caused by exposure of the cells to 20 mM lactate solution. When 0.25 mM phloretin was applied to these cells in addition to 2-TPGA, MCT1 transport was blocked and no significant pH change was detected. The differences between these two curves clearly show that 2-TPGA is a substrate for, and is actively transported by, MCT1.

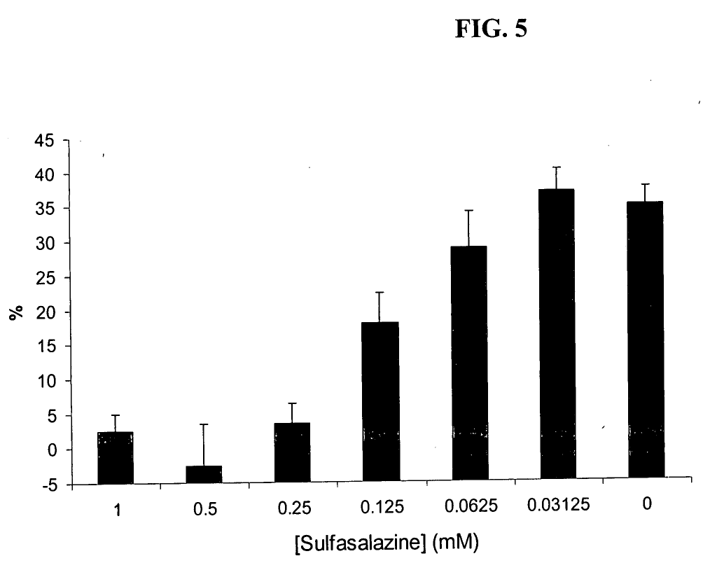

FIG. 5 shows the pH response of 2 mM lactate in the presence of various concentrations of sulfasalazine. FIG. 5 is a graph of sulfasalazine concentration ([sulfasalazine]) versus the relative percent decrease in intracellular pH compared to a standard decrease in intracellular pH caused by exposure of the cells to 20 mM lactate solution. As the concentration of sulfasalazine was increased, the relative intracellular pH decreased, indicating that sulfasalazine is an inhibitor of MCT1.

One disadvantage of the HEK pH assay is that it only reflects MCT1 transport. To test additional cloned MCT transporters, a pH assay was developed using Xenopus oocytes. Rather than using pH sensitive dyes, an electrophysiological assay for detecting intracellular pH was used in this assay. It is known that ROMK, a potassium channel in the kidney, is strongly sensitive to intracellular pH. The channel is completely open at normal intracellular pH (7.4) and is completely closed when the intracellular pH falls to 6.5. ROMK is unaffected by extracellular pH. Thus, the intracellular pH can be indirectly measured by measuring the net negative electric current flowing across the cell membrane (which negative current is caused by influx of potassium ions into the cell) when the cells are placed in a potassium ion containing medium such as a potassium buffer solution.

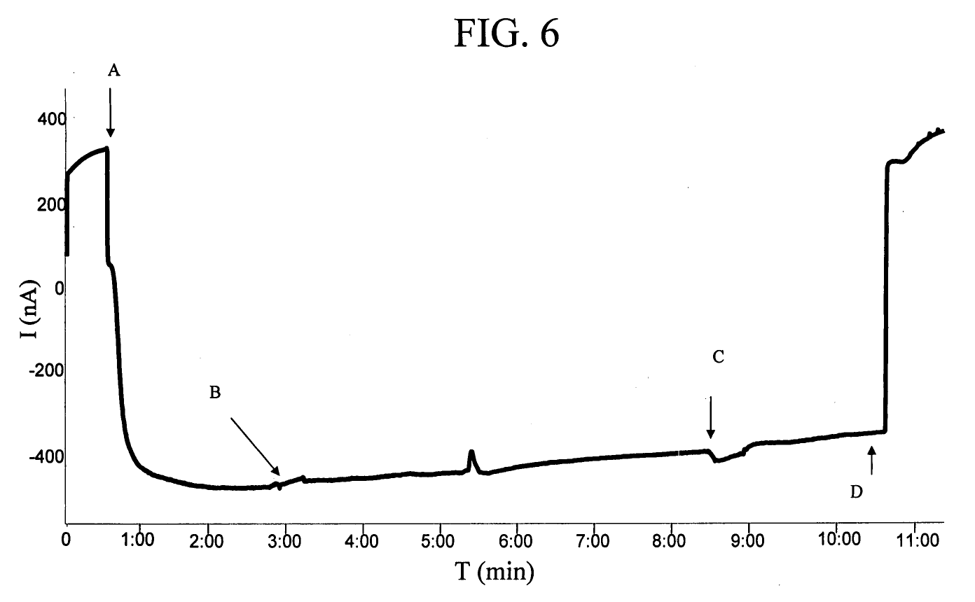

The complete open reading frame was cloned into a Xenopus oocyte expression plasmid, linearized, and cRNA was generated by run-off transcription using the T7 polymerase. Xenopus oocytes were prepared and maintained as previously described (Collins, et al., 1997) and injected with 1-30 ng RNA. Transport currents were measured 2-5 days after injection using two-electrode voltage-clamp (Axon Instruments). All experiments were performed using a modified oocyte Ringers solution (90 mM NaCl, 2 mM KCl, 1.8 mM CaCl₂, 1 mM MgCl₂, and 10 mM NaHEPES, pH 6.8). For experiments measuring ROMK currents, the above Ringer solution was modified to include 40 mM KCl. The membrane potential of oocytes was held between −30 and −80 mV and current traces acquired using PowerLab software. ROMK potassium currents were measured by raising the potassium concentration in the perfusion buffer from 2 mM to 40 mM. MCT1 and MCT4 were co-expressed with ROMK in oocytes by co-injection of cRNA. Application of an MCT1 or MCT4 substrate results in an intracellular acidification which inhibits ROMK potassium currents, resulting in an outward current. This outward current is not observed in oocytes that do not express ROMK or in the absence of extracellular potassium. The specificity of the currents is further determined by co-application of a non-transported MCT inhibitor (sulfasalazine or phloretin) to block the outward currents. To allow higher throughput application of compounds, the increase in the slope of the outward currents during the first 60 seconds of drug application are measured rather than the differential current induced by addition of the MCT substrate. Data are expressed as a percentage of the response to saturating lactate responses (change in the slope). The measured electrical current across the cell membranes of the MCT and ROMK transfected oocytes is shown in FIG. 6. The oocytes were exposed to a potassium ion-containing buffer (modified frog Ringers solution containing 100 mM KCl) beginning at time 24 sec (see arrow A in FIG. 6) which resulted in a large potassium selective “negative” current from about time 1-4 min. A 10 mM lactate solution was added at time 4:20 min (see arrow B in FIG. 6) which caused a reduction in the level of negative current. The lactate solution was allowed to incubate until time 5 min and then was washed out with the potassium ion-containing buffer, causing the level of negative current to return to the levels measured immediately before addition of the lactate solution. At time 7:30 min, the oocytes were exposed to 350 μM phloretin solution for 120 seconds. At time 9:19, the oocytes were exposed to a 350 μM phloretin and 10 mM lactate solution for 40 seconds (see arrow C in FIG. 6). At time 10:19 min (see arrow D in FIG. 6), the oocytes were exposed to a lower concentration potassium buffer solution (standard frog Ringers solution containing 2 mM KCl), which caused the negative current to stop. This portion of the FIG. 6 curve shows that when oocytes that co-express ROMK and either MCT1 or MCT4 are exposed to 10 mM lactate solution, a strong inhibition of the potassium current results. Co-application of phloretin with lactate completely blocks the effect of lactate on the potassium current. These results indicate that MCT transport acidifies the oocyte, and inhibits ROMK.

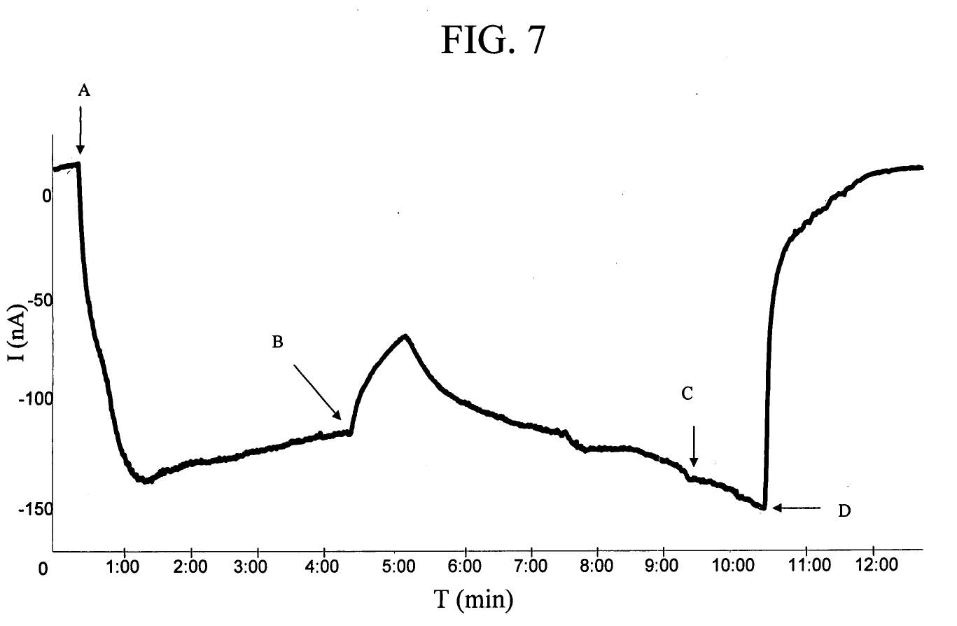

This can be compared with the curve shown in FIG. 7 which was generated under similar conditions using oocytes that express ROMK but neither MCT1 or MCT4. In FIG. 7, exposure of the oocytes to high concentration potassium ion buffer was conducted from time 28 sec (see arrow A in FIG. 7) through time 10:21 min (see arrow D in FIG. 7). Over this period of time, substantially no change in the negative potassium current was observed at either the time of addition of 10 mM lactate solution (see arrow B in FIG. 7) or at the time of addition of 10 mM lactate and 350 μM phloretin solution (see arrow C in FIG. 7).

Application of different concentrations of lactate indicate that ROMK responses can be detected at lower lactate concentrations in the oocyte assay than in the HEK pH assay. The oocyte MCT pH assay is a useful method for identifying novel MCT substrates for a variety of cloned MCT proteins.

Transport and Cytotoxicity Studies of Two Nitrogen Mustard Compounds

The transport and cytotoxicity of two nitrogen mustard compounds, 3-bis(2-chloroethyl)amino-4-methoxybenzoic acid and bis(2-chloroethyl)aminoxyacetic acid, were tested in Xenopus oocytes and in four human cancer cell lines. These compounds were synthesized as described below.

3-bis(2-chloroethyl)amino-4-methoxybenzoic acid (Compound 1) was synthesized as follows: 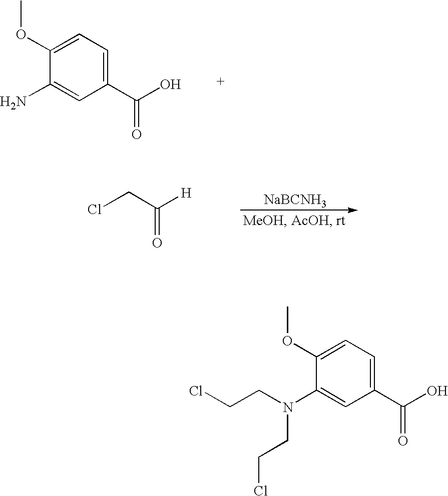
To a stirred solution of 3-amino-4-methoxybenzoic acid (0.85 g, 0.5 mmol) in 25 ml methanol at ice-bath temperature, chloroacetaldehyde (9 ml, 4 mmol) was added slowly as an approximately 50% aqueous solution. After stirring for 15 min, sodium cyanoborohydride (1.57 g, 2.5 mmol) was added slowly into the reaction mixture. The resulting mixture was acidified (approximately pH 6) using acetic acid and further stirred at room temperature for 2 hours. The progress of the reaction was monitored by thin layer chromatography (tlc) and liquid chromatography/mass spectrometry (LC/MS). The reaction mixture was concentrated in a rotavapor at room temperature under vacuum. The residue was diluted with ethyl acetate, and washed with 5% HCl solution and water. The organic layer was dried over Na₂SO₄ and evaporated. The residue was recrystalized using dichloromethane and hexane to obtain pure 3-bis(2-chloroethyl)amino-4-methoxybenzoic acid as a white powder in 79% (1.15 g) yield. The resulting compound was characterized by ¹H NMR spectrometry. The result of the characterization was as follows: 1H NMR (CDCl₃, 400 MHz): δ3.53 (8H, m); 7.03 (1H, d, J=8.4 Hz); 7.68 (1H, d, J=2.4 Hz), 7.74 (1H, dd, J=2.4 and 8.4 Hz). MS (ESI): m/z 292.41 (M+H)⁺ and 290.35 (M−H)⁻.

Bis(2-chloroethyl)aminoxyacetic acid (Compound 2) was synthesized as follows: 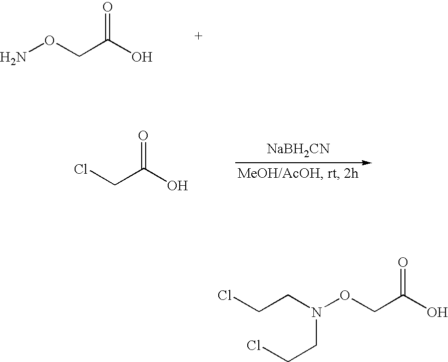
To a stirred solution of carboxymethoxylamine hemihydrochloride (0.54 g, 0.5 mmol) in 25 ml methanol at ice-bath temperature, chloroacetaldehyde (9 ml, 4 mmol) was added slowly as an approximately 50% aqueous solution. After stirring for 15 min, sodium cyanoborohydride (1.57 g, 2.5 mmol) was added slowly into the reaction mixture. The resulting mixture was acidified (approximately pH 6) using acetic acid and further stirred at room temperature for 2 hours. The progress of the reaction was monitored by tlc and LC/MS. The reaction mixture was concentrated in a rotavapor at room temperature under, vacuum. The residue was diluted with ethyl acetate and washed with 5% HCl solution and water. The organic layer was dried over Na₂SO₄ and evaporated. The residue was purified by preparative LC/MS to obtain pure bis(2-chloroethyl)aminoxyacetic acid as a white powder in 55% (0.59 g) yield. The resulting compound was characterized by ¹H NMR spectrometry. The result of the characterization was as follows: 1H NMR (CDCl₃, 400 MHz): δ3.21 (4H, t, J=6.4 Hz); 3.71 (4H, t, J=6.4 Hz); 4.48 (1H, s). MS (ESI): m/z 216.13 (M+H)⁺ and 214.10 (M−H)⁻.

The transport of Compounds 1 and 2 was tested on oocytes expressing human MCT1 (hMCT1 ) and in non-expressing cells (i.e., not transfected with human MCT1 RNA). Xenopus oocytes were injected with hMCT1 RNA as generally described above in the section entitled Studies of Cloned MCT Transporters. Briefly, two days after injection, both injected oocytes and non-injected control oocytes were prepared in ND96 buffer, pH 6.8 and exposed to 500 μM of Compounds 1 and 2 for 5 minutes. Each concentration of compound was tested in triplicate with four oocytes per condition. After five minutes, each set of oocytes was washed four times with 0.5% BSA ND96 (supra) using vertically consecutive wells of a 24-well plate at one minute intervals. After the final wash, each set of oocytes was manually lysed in an Eppendorf tube containing 80% methanol. The Eppendorf tubes containing each lysate were spun at 13 k rpm for 10 mins, and then the supernatant was injected into the LC/MS/MS for determination of the uptake of Compounds 1 and 2. The concentration of Compounds 1 and 2, as applicable, in each lysate was determined by comparison with a standard curve for the respective compound.

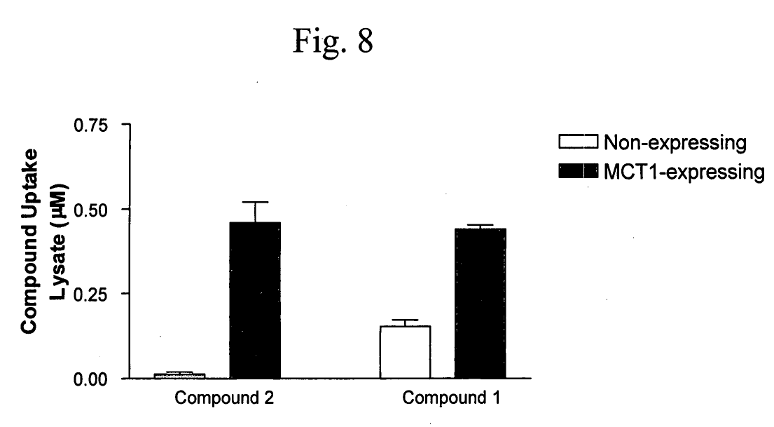

Oocytes expressing hMCT1 exhibited higher levels of uptake of both compounds, as compared with non-expressing cells. As shown in FIG. 8, oocytes expressing hMCT1 contained about 50 μM of the compounds, as compared with lower amounts in the non-hMCT1 expressing controls, indicating the compounds were transported into the oocytes by the hMCT1 transporter.

The cytotoxicity of Compounds 1 and 2 was assessed in four human cancer cell lines that express high levels of the MCT1 transporter, a HT29 human colon carcinoma cell line, a KB human oral epidermoid carcinoma cell line, a LoVo human colorectal cancer cell line, and a PC3 human prostate cancer epithelial cell line. HT29, KB, LoVo, and PC3 cells were seeded on UV sterilized, black, clear-bottom 96-well plates at approximately ten thousand cells per well. The cells were allowed to adhere overnight at 37° C., 5% CO₂ in a humidified environment in the following media: for HT-29cells, RPMI 1640 with 25 mM HEPES and 2 mM L-glutamine, supplemented with 5% FBS, 2 mM L-glutamine, and Pen/Strep/Fungizone; for LoVo and PC-3 cells, F12K supplemented with 10% FBS, 2 mM L-glutamine and Pen/Strep/Fungizone; and for KB cells, a 1:1 mixture of HYQ SFM4HEK293 (Hyclone):RPMI 1640 with 25 mM HEPES and 2 mM L-glutamine, supplemented with 2.5% FBS, 2 mM L-glutamine and Pen/Strep/Fungizone. The following day, serial dilutions of Compounds 1 and 2 were added to the cells, in duplicate, starting with a concentration of 1 mM to about 1 μM in the culture medium for HT29 cells. In addition, each plate contained a row of positive controls, a row of media only controls, and a row of negative controls in which 100% DMSO was added. The positive control for cytotoxicity was chlorambucil. The positive controls for transport were lactic acid and thiophene glyoxylic acid. After the compounds and controls were added to the cells, the plates were returned to the cell culture incubators. After 48 hours, 10 μl Alamar blue was added to each well. Fluorescence of each well was read six hours after adding the Alamar blue. The percentage of live cells was calculated for each well by the following formula: {Fluor_{Well}−Fluor_{NegControl})/Fluor_{PosControl}−Fluor_{NegContol})}*100. GI50 values (the concentration of the compound at which 50% of the cells exhibited growth inhibition; in μM) were determined by plotting the percentage of live cells at each compound concentration versus the concentration of each compound, and determining the concentration at which 50% of the cells exhibited growth inhibition.

The following table shows the GI50 values for Compounds 1 and 2. Both compounds exhibited cytotoxicity in the four human cancer cell lines tested. The GI50 values for Compound 2 ranged from about 15 to about 116 μM. The GI50 values for Compound 1 ranged from about 205 to about 1024 μM.

GI50 (μM) 48 hour treatment

| Compound | HT29 | KB | LOVO | PC3 |
|---|---|---|---|---|
| 1 | 1024 | 401 | 205 | 242 |
| 2 | 116 | 102 | 30 | 15 |

Although the foregoing compounds, conjugates and methods have been described in detail for purposes of clarity of understanding, it will be obvious that certain modifications may be practiced within the scope of tonv claim(s) granted herefrom. All publications and patent documents cited herein are hereby incorporated by reference in their entirety for all purposes to the same extent as if each were so individually denoted.

1. A method of screening agents, conjugates or conjugate moieties for activity useful for treating or diagnosing cancer, comprising
providing a cell expressing an MCT1 or MCT4 transporter, the transporter being situated in the plasma membrane of the cell;
contacting the cell with an agent, conjugate or conjugate moiety; and
determining whether the agent, conjugate or conjugate moiety passes through the plasma membrane via the transporter;
wherein the agent, conjugate or conjugate moiety comprises a cytotoxic or imaging component or the method further comprising linking the agent, conjugate or conjugate moiety to a cytotoxic or imaging component.

2. The method of claim 1, further comprising contacting the agent, conjugate, or conjugate moiety, with a cancerous cell and determining whether the agent kills or inhibits growth of the cell.

3. The method of claim 2, wherein the conjugate is 3-bis(2-chloroethyl)amino-4-methoxybenzoic acid or bis(2-chloroethyl)aminoxyacetic acid.

4. The method of claim 1, wherein the cancerous cell is present in an animal.

5. The method of claim 1, wherein the cytotoxic component is selected from platinum or nitrogen mustard.

6. The method of claim 1, wherein the agent, conjugate or conjugate moiety comprises a monocarboxylate group.

7. The method of claim 1, wherein if cell expresses the transporter MCT1, the cancerous cell is other than a brain cell or melanoma and if cell expresses the transporter MCT4, the cancerous cell is other than a melanoma.

8. The method of claim 1, further comprising administering the agent, conjugate or conjugate moiety to an undiseased animal and determining any toxic effects.

9. The method of claim 1, wherein the cell is an oocyte or a HEK cell.

10. (Canceled)

11. (Canceled)

12. (Canceled)

13. (Canceled)

14. (Canceled)

15. A pharmaceutical composition comprising a cytotoxic or imaging agent linked to a conjugate moiety to form a conjugate, wherein the conjugate moiety has a higher Vmax for MCT1 or MCT4 than the cytotoxic agent alone.

16. The pharmaceutical composition of claim 15, wherein the conjugate moiety has at least 5 times the Vmax for MCT1 or MCT4 as the cytotoxic or imaging agent alone.

17. The pharmaceutical composition of claim 15, wherein the conjugate moiety has a Vmax for MCT1 that is at least about 1% of the Vmax of lactate for MCT1.

18. The pharmaceutical composition of claim 15, wherein the conjugate moiety has a Vmax for MCT1 that is at least about 50% of the Vmax of lactate for MCT1.

19. The pharmaceutical composition of claim 15, wherein the conjugate moiety has a Vmax for MCT4 that is at least about 1% of the Vmax of lactate for MCT4.

20. The pharmaceutical composition of claim 15, wherein the conjugate moiety has a Vmax for MCT4 that is at least about 50% of the Vmax of lactate for MCT4.

21. The pharmaceutical composition of claim 15, wherein the cytotoxic agent is 3-bis(2-chloroethyl)amino-4-methoxybenzoic acid or bis(2-chloroethyl)aminoxyacetic acid.

22. A method of formulating a cytotoxic or imaging agent, comprising:
linking the cytotoxic or imaging agent to a conjugate moiety to form a conjugate, wherein the conjugate moiety has a greater Vmax for an MCT1 and/or MCT4 transporter than the agent alone; and
formulating the conjugate with a pharmaceutical carrier as a pharmaceutical composition.

23. The method of claim 22, wherein the conjugate is 3-bis(2-chloroethyl)amino-4-methoxybenzoic acid or bis(2-chloroethyl)aminoxyacetic acid.

24. A method of delivering a cytotoxic or imaging agent, comprising
administering to a patient a pharmaceutical composition comprising a cytotoxic or imaging agent linked to a conjugate moiety to form a conjugate, wherein the conjugate has a higher Vmax for an MCT1 and/or MCT4 transporter, whereby the conjugate or agent, after cleavage of the conjugate moiety, passes through the transporter into the cancerous cells in the patient.

25. The method of claim 24, wherein the Vmax of the conjugate is at least two-fold higher than that of the agent alone.

26. The method of claim 24, wherein the cancerous cells are present in a solid tumor.

27. The method of claim 24, further comprising determining a level of expression of MCT1 and/or MCT4 in the cancerous cells in excess of a level in noncancerous cells from the same tissue.

28. The method of claim 24, wherein the cytotoxic agent is a nitroimidizole and the method further comprises irradiating the patient to kill cancerous cells that have taken up the conjugate.

29. (Canceled)

30. A method of treating cancer, comprising
administering to a patient suffering from cancer an effective amount of an inhibitor of an MCT1 and/or MCT4 transporter, whereby the inhibitor inhibits transport through MCT1 and/or MCT4 transporters thereby lowering the intracellular pH within cells of the cancer;
administering a chemotherapeutic agent that that exhibits greater toxicity at lower intracellular pH, thereby, in combination with the inhibitor, killing or inhibiting the growth of the cells of the cancer with lower intracellular pH.

31. The method of claim 30, wherein the chemotherapeutic agent is selected from the group consisting of lonidamide, sulfur-containing platinum compounds, and camptothecin analogs.

32. The method of claim 31, wherein the chemotherapeutic agent is lonidamide.

33. The method of claim 30, wherein the cancer cells are present in a solid tumor.

34. (Canceled)

35. A method for screening an agent for capacity to inhibit a cotransporter of a proton and a substrate, comprising
providing a cell expressing an ion channel that is inhibited by reduction in intracellular pH and the co-transporter;
contacting the cell with a known substrate of the transporter and an agent;
measuring current across the cell membrane relative to the current when the cell is contacted with the known substrate in the absence of the agent, wherein an increase in current indicates the agent inhibits transport of the substrate thereby inhibiting intracellular acidification of the cell and reducing inhibition of the ion channel.

36. The method of claim 35, wherein the co-transporter is MCT1 and/or MCT4.

37. The method of claim 35, wherein the ion channel is ROMK.

38. The method of claim 35, wherein the known substrate is lactate.

39. A method for screening an agent, conjugate or conjugate moiety for capacity to be a substrate for a cotransporter of a proton and a substrate, comprising
providing a cell expressing an ion channel that is inhibited by reduction in intracellular pH and the co-transporter;
contacting the cell with the agent, conjugate or conjugate moiety;
measuring current across the cell membrane relative to the current in the absence of an agent, wherein a decrease in current indicates the agent is a substrate of the co-transporter, whereby uptake of the agent, conjugate or conjugate moiety increases intracellular acidification of the cell and inhibits of the ion channel.

40. The method of claim 39, wherein the co-transporter is MCT1 and or MCT4.

41. The method of claim 39, wherein the ion channel is ROMK.

42. A cell expressing a co-transporter of protons and a substrate and a pH-sensitive ion channel, the co-transporter and ion channel being situated in the plasma membrane of the cell, wherein the co-transporter and/or ion channel is encoded by a nucleic acid transformed into the cell.

43. A cell line comprising a population of cells as defined in claim 39.

44. A method for measuring intracellular pH, comprising
providing a cell expressing an ion channel that is inhibited by reduction in intracellular pH, the ion channel being situated in the plasma membrane of the cell;
monitoring current across the membrane,
determining a measure of intracellular pH from the current, wherein intracellular pH increases with the current.

45. The method of claim 44, wherein the ion channel is ROMK.

46. A method for measuring flux of an organic ion co-transported with a proton by a co-transporter, comprising:
providing a cell expressing an ion channel that is inhibited by reduction in intracellular pH and the co-transporter, the ion channel and the co-transporter being situated in the plasma membrane of the cell;
contacting the cell with an organic ion;
monitoring current across the membrane,
determining a measure of the flux of the organic ion wherein the flux of the organic ion is inversely related to the current across the membrane.

47. The method of claim 46, wherein the ion channel is ROMK.

48. The method of claim 46, wherein the co-transporter is a monocarboxylate transporter.

49. The compound 3-bis(2-chloroethyl)amino-4-methoxybenzoic acid.

50. The compound bis(2-chloroethyl)aminoxyacetic acid.
